(19) United States
(12) Patent Application Publication
(10) Pub. No.: US 2018/0008523 A1
(43) Pub. Date: Jan. 11, 2018

(54) ONYCHOMYCOSIS TREATMENT SYSTEM AND METHOD

(71) Applicant: DEVICEFARM, INC., Newark, CA (US)

(72) Inventors: Jeffrey N. Roe, Pleasanton, CA (US); Timothy C. Grammer, Pleasant Hill, CA (US); Raul Ignacio Barrera-Barraza, Fremont, CA (US)

(21) Appl. No.: 15/711,019

(22) Filed: Sep. 21, 2017

Related U.S. Application Data
(63) Continuation-in-part of application No. 15/701,649, filed on Sep. 12, 2017.
Application No. 15/701,649 is a continuation-in-part of application No. 15/464,761, filed on Mar. 21, 2017.
Application No. 15/464,761 is a continuation-in-part of application No. 14/963,552, filed on Dec. 9, 2015.
(60) Provisional application No. 62/089,945, filed on Dec. 10, 2014.

(51) Int. Cl. A61K 8/70; A61K 9/00; A61Q 3/02; A61Q 19/00

(52) U.S. Cl. CPC A61K 8/70; A61Q 3/02; A61K 9/0014; A61K 2800/81; A61Q 19/00

(57) ABSTRACT

A system and method includes a nonthermal plasma sub-system with a permeable UV filter for production and delivery of a redox gas solution to treat onychomycosis, wherein the redox gas solution comprises a reactive species dissolved in a perfluorocarbon liquid, and wherein the perfluorocarbon liquid is dispensed onto a gas-permeable membrane and wherein the reactive species may include, alone, or in combination, one or more of reactive oxygen, reactive nitrogen, reactive chlorine, or reactive bromine species, and the perfluorocarbon liquid may include perfluorodecalin.

### CROSS-REFERENCE TO RELATED APPLICATION

This application is a continuation-in-part of pending application Ser. No. 15/701,649 , filed Sep. 12, 2017, and is relatFed to and claims priority from, pending U.S. patent application Ser. No. 15/464,761, filed Mar. 21, 2017, and pending U.S. patent application Ser. No. 14/963,552, filed Dec. 9, 2015, which is related to and claims priority from US provisional patent application serial number 62/089,945, filed Dec. 10, 2014, entitled Onychomycosis Treatment Apparatus and Method, and pending U.S. patent application Ser. No. 15/674,163 filed Aug. 10, 2017, entitled Onychomycosis Treatment Apparatus and Method, and pending U.S. patent application Ser. No. 15/674,141 filed Aug. 10, 2017, entitled Onychomycosis Treatment Apparatus and Method, and pending U.S. patent application Ser. No. 15/674,120 filed Aug. 10, 2017, entitled Onychomycosis Treatment Apparatus and Method each of which is hereby incorporated by reference herein for all purposes.

### STATEMENT REGARDING FEDERALLY SPONSORED RESEARCH OR DEVELOPMENT

This invention was made with government support under NSF Award ID 1343994 and NIH Award ID 1R43GM112196-01 awarded by the National Science Foundation and the National Institute of Health. The government has certain rights in the invention.

### FIELD OF DISCLOSURE

The invention in one aspect relates generally to a system and method for the treatment of tissue infected with onychomycosis.

### BACKGROUND

Onychomycosis (also known as “dermatophytic onychomycosis,” or “tinea unguium”) is a fungal infection of the nail. It is the most common disease of the nails and constitutes about half of all nail abnormalities. This condition may affect toenails or fingernails, but toenail infections are particularly common It occurs in about 10% of the adult population.

The most common, symptom of a fungal nail infection is the nail becoming thickened and discolored. As the infection progresses, the nail can become brittle with pieces breaking off or coming away from the toe or finger. If left untreated, the skin can become inflamed and painful underneath and around the nail. There may also be white or yellow patches on the nail bed or scaly skin next to the nail, and an odor may result. Many times, the tissue surrounding the affected nail has inflammation and treatment of that tissue is also desirable. There is usually no pain or other bodily symptoms, unless the disease is severe, People with onychomycosis may experience significant psychosocial problems due to the appearance of the nail, particularly when fingers—which usually are always visible—are affected.

Dermatophytids are fungus-free skin lesions that sometimes form as a result of a fungus infection in another part of the body. This could take the form of a rash or itch in an area of the body that is not infected with the fungus. Dermatophytids can be thought of as an allergic reaction to the fungus.

The causative pathogens of onychomycosis include dermatophyt Candida, and non-dermatophytic molds. Dermatophytes are the fungi most commonly responsible for ortychomycosis in the temperate western countries; while Candida and nondermatophytic molds are more frequently involved in the tropics and subtropics with a hot and humid climate.

Trichophyton rubrum (T. rubrum) is the most common dermatophyte involved in onychomycosis. Other dermatophytes that may be involved are T. interdigitale, Epidermophyton floccosum, T violaceum, Microsporum gypseum, T. tonsurans, and T. soudanense. A common outdated name that may still be reported is Trichophyton mentagrophytes for T. interdigitale. The name T. mentagrophytes is now restricted to the agent of favus skin infection of the mouse; though this fungus may be transmitted from mice and their dander to humans, it generally infects skin and not nails.

Other causative pathogens include Candida and nondermatophytic molds, members of the mold genus Scytalidium (name recently changed to Neoscytalidium), Scopulariopsis, and Aspergillus. Candida mainly causes fingernail onychomycosis in people whose hands are often submerged in water. Scytalidium mainly affects people in the tropics, though it persists if they later move to areas of temperate climate.

All causative pathogens are susceptible to certain toxic gasses, such as ozone, oxides of nitrogen, and similar reactive materials. It is understood that fluid and solid materials may also have similar beneficial anti-pathogenic properties. However, there are a number of problems associated with the use of such anti-pathogenic substances to treat onychomycosis. The nail bed itself can act as a barrier to curative gasses and beneficial anti-pathogenic substances. Additionally, the curative gasses can cause inflammation to the tissue surrounding the affected nail and the inflammation is a negative side effect of the treatment. Thus, there remains a need for a system and method for the treatment of onychomycosis that generates the curative gasses and beneficial anti-pathogenic substances and permits substances to traverse, surround and/or enter the nail bed and similar physiological structures for a beneficial effect while mitigating the effect of inflammation to the surrounding tissue

There also remains an unmet medical need for a topical treatment device, and treatment method for onychomycosis that is effective, short in duration of treatment, and reduces inflammation in the surrounding skin without the undesirable side effects of the prior art. Many chemical compounds exhibit antifungal (fungistatic or fungicidal) properties and can be incorporated into creams, lotions, gels, solutions and the like. However, antifungal compounds applied topically (i.e., directly to the nail) do not adequately and consistently penetrate the nail bed to kill the fungus at its source, and thus are not consistently effective. Also, they can irritate the surrounding skin, so a means to minimize the effect of inflammation in the surrounding skin is needed.

A method to capture the curative gasses and beneficial anti-pathogenic substances generated so that they do not escape into the surrounding environment is needed so the gases do not escape from the generating and treatment device.

Thus, an additional improved apparatus and method for effectively treating onychomycosis is desirable.

### SUMMARY

The present disclosure provides a system and method that includes delivery of a redox gas solution to treat onychomycosis, wherein the redox gas solution comprises a reactive species dissolved in a perfluorocarbon liquid.

In one exemplary embodiment, the reactive species may include, alone or in combination, one or more of reactive oxygen, reactive nitrogen, reactive chlorine, or reactive bromine species. The perfluorocarbon liquid may include perfluorodecalin.

In one exemplary embodiment, a system is provided that may include a housing with a chamber disposed therein. The chamber may include an opening through which a foot or hand to be treated may be at least partially inserted. One or more reactive species generators may be disposed within the chamber. In one embodiment, the toes of the inserted foot or nails of the hand may be positioned a desired distance from the one or more reactive species generators. A disposable tray may be used to help prevent contact between the foot or hand and the sides (i.e., bottom, walls) of the housing chamber. In one embodiment, the disposable tray may include a curtain that closes the opening about the inserted foot or hand, e.g., to help prevent the escape of reactive species through the opening. During treatment, the generators provide reactive species to the chamber containing the inserted foot or hand for a desired period of time.

In one exemplary embodiment, the device utilizes a nonthermal plasma subunit containing a permeable ultraviolet light (UV) filter built into a plasma system to prevent damage to the patients' skin surrounding the infection. The UV filter limits the UV exposure to the skin that could otherwise cause skin damage. The skin damage that can occur is an acute inflammatory response of skin to exposure to ultraviolet radiation (UVR). UVR causes vasodilation and release of mast cell mediators, leading to an inflammatory response.

In one exemplary embodiment, the device utilizes a nonthermal plasma subunit containing a permeable UV filter built into a plasma system to prevent damage to the patients' skin surrounding the infection. The permeable UV filter is treated to improve the UV blocking capabilities of the fabric and can also be coated with anti-fungal compounds to protect the fabric from patient cross contamination and improve the fungal killing effectiveness of the instrument.

In one exemplary embodiment, carbon nanotubes are utilized as a means to increase plasma creation at the wire mesh ground electrode where the plasma gas is created, thereby increasing the effectiveness of the treatment.

The generation rate of plasma depends on the local electric field strength. The plasma discharge is strongest in regions with larger electric field strength, such as near a curved surface like that of a needle-thin wire or cutting edge Carbon nanotubes grown on the ground electrode provide multiple points where high electric field strengths are achieved. In the region of high electric field strength, the condition for a high voltage gas breakdown is fulfilled and plasma is created.

In one exemplary embodiment, the device uses a venting louver as a means to enhance plasma treatment of nails by concentrating the plasma reactive species over the nail and improving fungal killing within the nail. Plasma gas takes longer to penetrate the nails than it does the skin surface due to the thickness and density of the nail barrier to diffusion of the plasma gas. A venting louver (i.e., a combination component of (1) a physical barrier that blocks plasma transport and (2) venting holes in the barrier that allow for plasma transport), can optimize treatment efficiency. A louver is a set of angled or flat strips in an opening or screen to allow air or light to pass through the holes or slot formed by the angled or flat strips. A venting louver allows the treatment system to concentrate the plasma as much as possible over the nails relative to the skin by positioning the holes in the louver above the target nails. The angle of the strips may be adjustable or fixed to form the holes and vary the size of the holes. Also the louver divides the chamber into two sub-chambers; one sub-chamber contains the ground electrode of the plasma screen. The second sub-chamber contains the body part to be treated. The venting louver thereby acts as a barrier blocking some portion of the plasma screen. Optionally, the holes can be covered with a permeable UV filter/blocking fabric which reduces the UV exposure to the patient,

In one exemplary embodiment, the perfluorocarbon liquid is pre-loaded into a toe cot or elastic sleeve. These sleeves are placed onto the patient's infected toe(s) to cover the infected toenails before the foot is slipped into the plasma treatment chamber. The sleeve positions the pre-loaded perfluorocarbon liquid in close proximity to the infected nail. At least the portion of the sleeve that covers the nail and contains the perfluorocarbon liquid must be penetrable by the plasma so as to allow for plasma flow into and concentrate around the sleeve area above the infected nail. The part of the sleeve that covers the skin of the toe can either be made permeable or impermeable to plasma depending on whether it is desired to treat or protect the skin, respectively.

In another exemplary embodiment, the chamber, of the treatment instrument may include a disposable gas-permeable membrane installed on a dispensing reel and a take-up reel and a syringe having perfluorocarbon solution. The membrane would be positioned between one or more reactive species generators and the toes of the inserted foot or nails of the hand. In one embodiment, the nails of the inserted foot or hand may be positioned so that they touch the membrane that is imbibed or saturated with perfluorocarbon solution. Before start of treatment, a syringe disperses perfluorocarbon solution onto the membrane so that the solution imbibes or saturates the membrane such that the nails become coated with perfluorocarbon. The syringe can disperse additional perfluorocarbon solution to the membrane during the treatment to allow for replenishment of the perfluorocarbon solution to the nails being treated.

In another exemplary embodiment, the chamber of the treatment instrument may include an exhaust device containing a filter and fan to capture the curative gasses and beneficial anti-pathogenic substances generated so that they do not escape into the surrounding environment. The exhaust devices is positioned to deactivate the reactive gases after treatment but before the body portion is removed from the treatment chamber. The exhaust device removes, the reactive gas from the device by pumping or blowing the gas from the device through a filter.

In another exemplary embodiment, a post treatment of acrylic or shellac is applied after the treatment with the curative gasses and beneficial anti-pathogenic substances generated to extending the nail degassing time and increase the exposure of the infection to the curative gasses and beneficial anti-pathogenic substances generated by sealing the upper portion of the nail.

Other benefits and advantages of the present disclosure will be appreciated from the following detailed description.

### BRIEF DESCRIPTION OF THE DRAWINGS

Exemplary embodiments of a system and method are shown in the accompanying drawings.

FIG. 1 is an illustration by flowchart of an exemplary method in accordance with the disclosure.

FIG. 2 is a cross-sectional view of a portion of an exemplary system in accordance with the disclosure.

FIG. 3A is a cross-sectional view of a container including a n exemplary perfluorocarbon liquid in accordance with the disclosure.

FIG. 3B is a cross-sectional view of a cap for the container shown in FIG. 3A, the cap including an applicator for providing perfluorocarbon liquid to a nail/tissue.

FIG. 3C is a perspective view of an exemplary application of perfluorocarbon liquid to nail/tissue using the applicator shown in FIG. 3B.

FIG. 4 is a partial schematic view of an exemplary plasma generating device proximate nail/tissue to which a perfluorocarbon liquid has been applied in accordance with the disclosure.

FIG. 5A is a perspective view of an exemplary toe-clip applicator in accordance with the disclosure.

FIG. 5B is a partial cross-sectional view of the exemplary toe-clip applicator shown in FIG. 5A.

FIG. 6 is a perspective view of an alternate exemplary treatment system embodiment in accordance with the disclosure.

FIG. 7 is a perspective view of another alternate exemplary treatment system embodiment in accordance with the disclosure.

FIG. 8 is an assembly view of the exemplary treatment system shown in FIG. 7.

FIG. 9 is an assembly view of the chamber housing of the exemplary treatment system shown in FIG. 8.

FIG. 10A is an assembly view of the exhaust duct and filter cartridge of the exemplary treatment system shown in FIG. 8.

FIG. 106 is a diagram of the blower and filter cartridge shown in FIG. 10A.

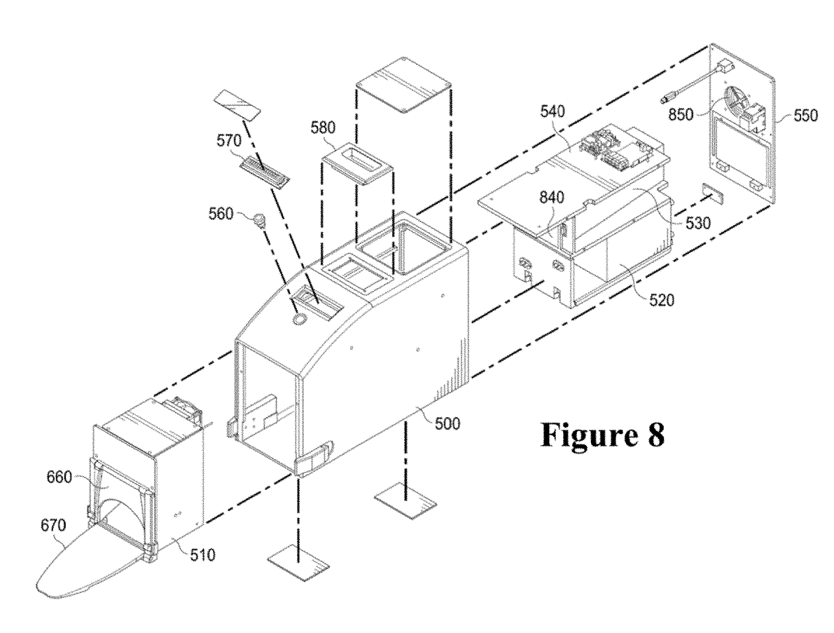

FIG. 11 is an assembly view of the power supply housing of the exemplary treatment system shown in FIG. 8.

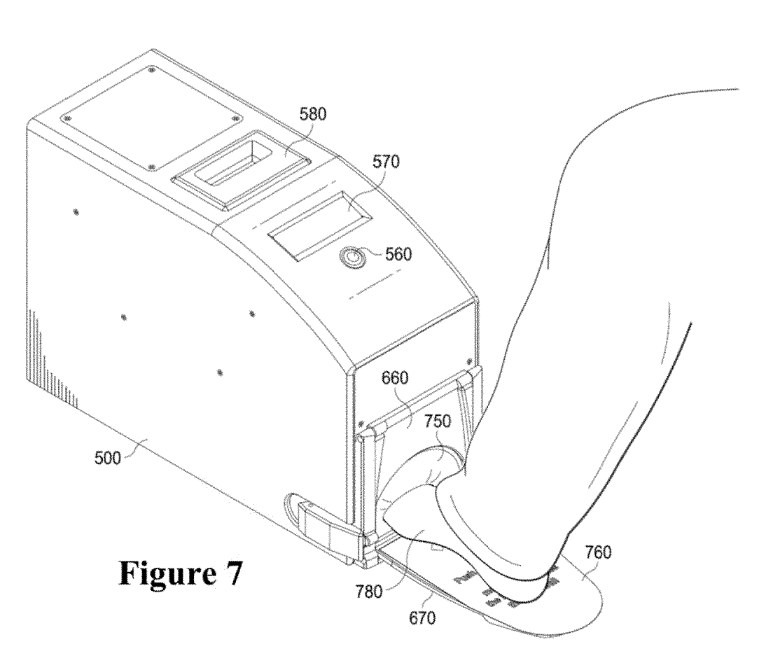

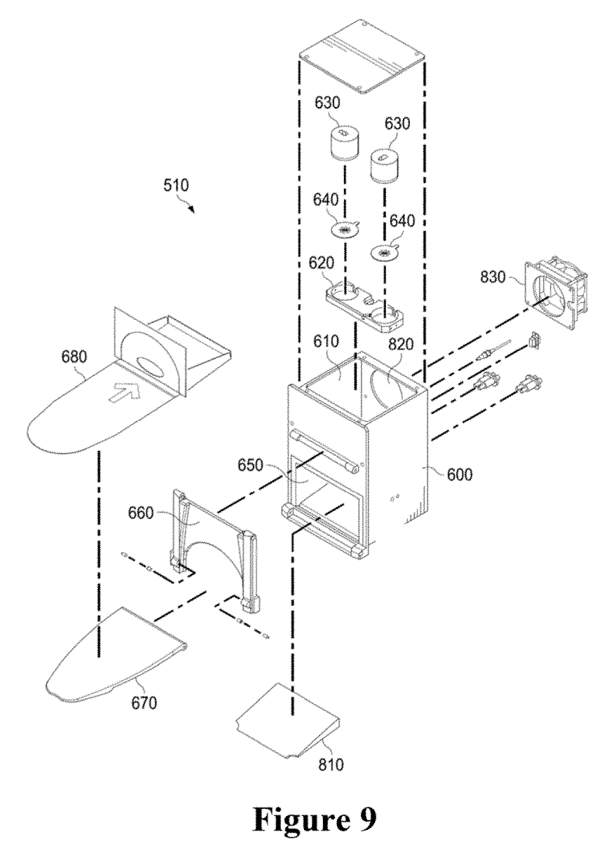

FIG. 12A is a perspective view of a disposable tray of the exemplary treatment system shown in FIGS. 7 and 9.

FIG. 128 is a perspective view of a disposable tray in a folded configuration for use in the exemplary treatment system shown in FIGS. 7 and 9.

FIG. 13 is a cross-sectional view of a container including an exemplary nanoparticle TiO₂ powder suspended in an alcohol and glycol mixture, monopropylene glycol, or water with a neutral pH water.

FIG. 14 is a perspective view of the treatment instrument with a disposable transfer medium gas-permeable membrane installed on a dispensing reel and a take-up reel and a syringe having perfluorocarbon solution.

FIG. 15 is an exploded perspective view of a nonthermal plasma subunit of the invention.

FIG. 16 i a sketch showing a venting louver that enhances plasma treatment of nails

FIG. 17 is a perspective view of an exemplar application of an acrylic or shellac to nail/tissue using an applicator.

### DETAILED DESCRIPTION

A UV filter is defined within this invention to mean a filter which screens out UV radiation, but is permeable to the plasma gas.

Embodiments of the invention and various alternatives are described. Those skilled in the art will recognize, given the teachings herein, that numerous alternatives and equivalents exist that do not depart from the invention. It is therefore intended that the invention not be limited by the description set forth herein or below.

A louver is a set of angled or flat strips in an opening or screen to allow air or light to pass through the holes or slot formed by the angled or flat strips. A venting louver is defined as a mechanical device placed in the chamber to form two sub-chambers: one containing the ground electrode of the plasma screen and a second sub-chamber configured to contain and treat an object or body part. Thereby, the venting louver acts as a barrier for some portion of the plasma being generated in the sub-chamber containing the plasma screen from entering directly into the treatment sub-chamber. The shape of the venting louver can be designed such that the plasma species entering the treatment sub-chamber through the holes in the louver can concentrate over the nails relative to the skin. The angle of the strips forming the louver may be fixed or adjustable to vary the size and positioning of the holes. Optionally, the holes can be covered with a permeable UV filter/blocking fabric to allow plasma gas flow, but prevent UV light from passing through.

A blower and cartridge assembly is defined as a fan, and a suitable filter medium. The filter medium can be either a pleated paper or membrane filter or a single or multi-stage activated carbon filter contained in a cardboard support. The filter can also be a combination of both a pleated paper or membrane filter and a single or multi-stage activated carbon filter. The fan can be either a propeller style blade, squirrel cage blower, Axial-flow fans that have blades that force air to move parallel to the shaft about which the blades rotate, centrifugal fan similar to a squirrel cage blower, cross-flow fan, bellows, Coanda effect fan, or Electrostatic fluid accelerator.

One or more specific embodiments of the system and method will be described below. These described embodiments are only exemplary of the present disclosure. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, for clarity and convenience only, and without limitation, the disclosure (including the drawings) sets forth exemplary representations of only certain aspects of events and/or circumstances related to this disclosure. Those skilled in the art will recognize, given the teachings herein, additional such aspects, events and/or circumstances related to this disclosure, e.g., additional elements of the devices described; events occurring related to onychomycosis treatment etc. Such aspects related to this disclosure do not depart from the invention, and it is therefore intended that the invention not be limited by the certain aspects set forth of the events and circumstances related to this disclosure.

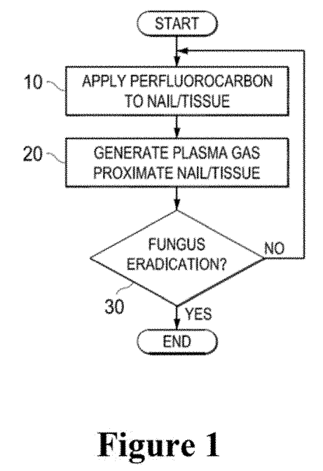

Turning now to the drawings, the figures show an exemplary treatment system and method. As described in FIG. 1, the system provides delivery of a redox gas solution to treat onychomycosis, wherein the redox gas solution comprises a reactive species dissolved in a perfluorocarbon liquid. The perfluorocarbon liquid is applied at step 10 to the nail/tissue. Then, at step 20, a plasma gas is generated proximate the nail/tissue. The plasma gas forms reactive species that dissolve in the perfluorocarbon liquid to form a redox gas solution. The process may repeat or continue until sufficient redox gas solution is produced to eradicate nail/tissue fungus, see step 30.

In one exemplary embodiment, the reactive species may include, alone or in combination, one or more of reactive oxygen, reactive nitrogen, reactive chlorine, or reactive bromine species. The reactive species may be formed through use of a non-Thermal plasma device or otherwise be provided.

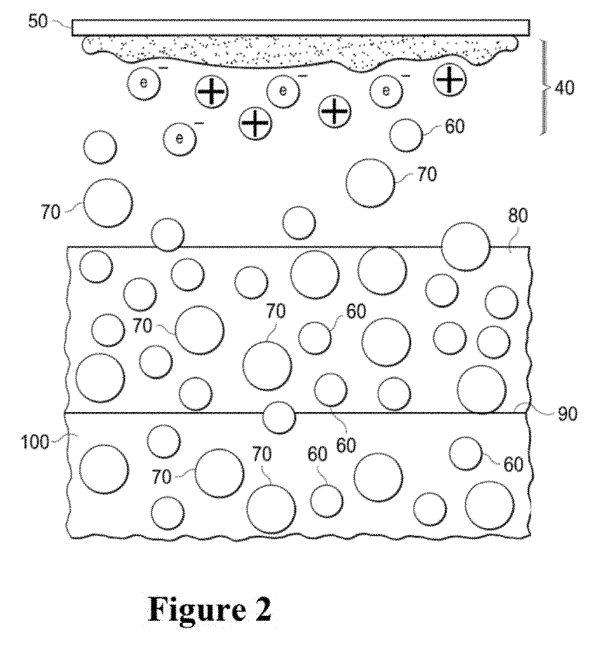

As shown in FIG. 2, a plasma 40 may be formed proximate a ground electrode 50. The plasma forms reactive species 60, 70. The reactive species 60, 70 dissolve in a perfluorocarbon liquid 80 applied to the surface 90 of nail/tissue 100. The redox gas solution including reactive species 60, 70 diffuses into the nail/tissue bed to eradicate fungus located therein. As shown in FIG. 2, the plasma generating device includes a ground electrode 50. In addition, the device generates a sufficiently high voltage signal applied between two electrodes, one of which is the ground electrode 50, where at least one of the electrodes is insulated by a dielectric material for plasma generation (not shown for clarity in FIG. 2).

### Perfluorocarbon Liquids (PFCs)/Other Facilitators

The perfluorocarbon liquid may include perfluorodecalin. Perfluorodecalin and other suitable perfluorocarbon liquids have desirable wetting, gas absorption and diffusion properties.

Perfluorocarbons (PFCs), fluorocarbons, or perfluorochemicals (terms which may be used interchangeably) liquids are formally derived from liquid hydrocarbons by replacing all the hydrogen atoms with fluorine atoms. This class of chemical compounds is characterized by its property to be extremely inert—chemically, biologically, and physiologically—due to the remarkable stability of the C—F bonds. The C—F bond is the strongest bond encountered in organic chemistry and its strength is further increased when several fluorine atoms are present on the same carbon atom. The presence of fluorine even reinforces the strength of the C—C bonds.

PFC liquids generally are clear, colorless, odorless, electrically non-conducting, and nonflammable. They are approximately twice as dense as water, and generally are capable of dissolving large amounts of physiologically important gases. For their gas uptake function, PFCs act only as a carrier of the gasses and do not react with the gas or produce the gases. PFCs are generally very chemically stable compounds that are not metabolized in body tissues. They are physiologically inert as there is no enzyme system capable of modifying liquid PFCs, neither metabolically nor catabolically. Liquid PFCs are both hydrophobic and lipophobic, i.e.,they are immiscible both with water and lipophilic liquids and generally form emulsions with them.

PFCs are used in a variety of industries. They were first synthesized in the 1920s and developed for industry in the 1940s. PFCs are currently being used in retinal detachment surgery, liquid ventilation therapy for the lungs, as a blood substitute and as ultrasound and radiological imaging agents. They are used in both cosmetics and paints to facilitate easier product spreading and in textile manufacturing as a fabric protector.

The terms suspension, mixture and solution, as used herein, are used interchangeably to mean a mixture of nano-powder in alcohol and glycol mixture, monopropylene glycol, or water with a neutral pH water.

The term “perfluorocarbon liquid” or “PFG liquid”, as used herein, may include organic compounds in which all (or essentially all) of the hydrogen atoms are replaced with fluorine atoms. Representative perfluorinated liquids include cyclic and non-cyclic perfluoroalkanes, cyclic and non-cyclic perfluoroamines, cyclic and noncyclic perfluoroethers, cyclic and non-cyclic perfluoroaminoethers, and any mixtures thereof.

The term “membrane” is defined as a gas permeable membrane composition that comprises at least one material selected from ceramics, polymers, woven substrates, non-woven substrates, polyamide, polyester, polyurethane, fluorocarbon polymers, polyethylene, polypropylene, polyvinyl alcohol, polystyrene, vinyl, plastics, metals, alloys, minerals, non-metallic minerals, wood, fibers, cloth, glass, and hydrogels.

Specific examples of perfluorinated liquid include the following: perfluoropentane, perfluorohexane, perfluoroheptane, perfluorooctane, perfluorodecalin, perfluoromethylcyclohexane, perfluorotributyl amine, perfluorotriamyl amine, perfluoro-N-methylmoφholine, perfluoro-N-ethylmoφholine, perfluoroisopropyl moφholine, perfluoro-N-methyl pyrrolidine, perfluoro-1,2-bis(trifluoromethyl)hexafluorocyclobutane, perfluoro-2-butyltetrahydrofuran, perfluorotriethylamine, perfluorodibutyl ether, and mixtures of these and other perfluorinated liquids.

Perfluorocarbons, in general,improve gas exchange and are a desirable medium to carry redox gases. PFC at one atmosphere of pressure can carry 20 times more oxygen than saline will hold. PFCs are low viscosity surfactants that may lower the surface tension on the nail, so the PFC may spread uniformly and quickly over the nail structure. The low surface tension contributes to improved wetting of the surfaces. The surface tensions of PFCs are generally less than 20 dynes/cm and usually between 10 to 20 dynes/cm when measured at 25° C. When used in lung injury for ARDS patients, surface tension in the lung is noted to be 67 to 75 dynes/cm. In a lung with PFC, the surface tension is only 18 dynes/cm, which helps prevent alveolar collapse and reduces alveolar opening pressures.

PFCs may displace water and circulate to those areas where gas exchange is diminished. PFCs also may wash out debris if the debris is lighter than the PFC used. PFCs are not taken up by the body and do not break down into toxic metabolites.

Perfluorocarbon liquids may be compounds containing a high level of carbon-bound fluorine that are liquid at or below 106° F. at atmospheric pressure. These fluorinated fluids may be capable of dissolving a substantial amount of a redox gas at operating conditions, typically in a temperature range from about 0° C. to about 50° C. The perfluorocarbon liquid may be converted in whole or in part to a redox gas solution before topical application by dissolving the reactive gaseous species into perfluorocarbon liquid at the manufacturing facility and delivering the topical composition to the customer in a usable form such that the customer can apply the solution to the infected area as a treatment. In one exemplary embodiment, PFC fluids may dissolve at least 500 mL of gaseous chlorine per 100 mL of fluid at 1 atm and 25° C. In another exemplary embodiment, the PFC fluids may dissolve at least 1200 mL of gaseous chlorine at 1 atm and 25° C. The oxidizing gas solutions used in the described methods may be saturated with a desired oxidizing gas. In another example, the concentration of ozone in the PFC may be greater than 1 ppm but less than 500 ppm. Fluorinert™ Fluids, product bulletin 98-0211-8301-1(65,05)R, issued 5/95, available from 3M Co., St. Paul, Minn., provides the solubility of many oxidizing gases in Fluorinert™ Electronic Fluids.

Other perfluorocarbons that may be used include, by way of example, perfluorocarbons such as fluoroheptanes, fluorocycloheptanes, fluoromethylcycloheptanes, fluorohexanes, fluorocyclohexanes, fluoropentanes, fluorocyclopentanes, fluoromethylcyclopentanes, fluorodimethylcyclopentanes, fluoromethylcyclobutanes, fluorodimethylcyclobutanes, fluorotrimethylcyclobutanes, fluorobutanes, fluorocyclobutanse, fluoropropanes, fluoroethers, fluoropolyethers, fluorotributylamines, fluorotriethylamines, perfluorohexanes, perfluoropentanes, perfluorobutanes, perfluoropropanes, sulfur hexafluoride, Methylperfluorobutylether (GransilSiW 7100) or Perfluoro(tert-butylcyclohexane). Also, mixtures of perfluorocarbons could also be utilized in this invention that combine different perfluorocarbons and perfluorocarbon compositions such as PFC emulsions or PFC gels.

Other liquids may fulfill the mechanism of penetration enhancer/gas carrier and could concentrate the antifungal species from the plasma. In addition to PFCs, alternative molecules that both concentrate and promote gas exchange may be used and include, but are not limited to, neuroglobin, apomyoglobin, hemoglobin, myoglobin, and synthetic blood or blood substitutes such as respirocytes.

To minimize damage to the surrounding skin local to the infection due to the exposure to UVR. A permeable UV filter can be incorporated as a component of the non-thermal plasma generator. The permeable UV filter minimizes skin damage to skin or tissue surrounding the infection by blocking the exposure to the potentially harmful UV radiation. Current plasma systems treat the infection for relatively short periods of time from 1-5 minutes and due to the short duration of exposure of the infection to the reactive species, they do not effectively treat the infection. However, you could not complete a 45-min plasma treatment without a UV filter placed between the plasma heads and the patient or else UV radiation damage to the surrounding tissue will occur. If this UV filter was not present the patient would receive high dosage of UVR in the region directly exposed to the plasma head and the skin surrounding the infection would become damaged similar to the effect of over exposure to the sun, which is commonly referred to as sunburn.

In another exemplary embodiment, the perfluorocarbon or other liquid may include a co-solvent to improve as desired specific physical properties of the fluid. A semifluorinated alkane (SFA) that has a non-fluorinated hydrocarbon segment may be added to a PFC liquid. In another exemplary embodiment, the liquid composition may comprise the combination of more than one PFC and/or more than one SFA. It may be useful to combine PFCs and SFAs to achieve a particular desired target property, such as a certain density, viscosity, lipophilicity or soluble capacity for a particular active ingredient such as a dye. The SFA may be essentially nonreactive with the redox gas. The SFA also may not reduce the solubility of the redox gas in the PFC. In one exemplary embodiment, one or more useful SFAs may be selected from a group of SFAs including F₄H₅, F₄H₆, F₄H₈, F₆H₆ and F₆H₈,

### Redox Gas Solutions

In one exemplary embodiment, the perfluorocarbon liquid absorbs antimicrobial substances generated by a plasma-generating device. See, e.g., FIG. 2. In one exemplary aspect, onychomycosis treatment is enhanced using a solution containing gaseous reactive oxygen or reactive nitrogen or reactive chlorine/bromine species that are dissolved in a perfluorocarbon liquid.

For convenience only and without limitation, a solution containing gaseous reactive oxygen or reactive nitrogen or reactive chlorine/bromine species dissolved in a perfluorocarbon liquid shall be referred to herein as a “redox gas solution.”

In accordance with another exemplary embodiment, an onychomycosis treatment system and method includes a topical composition to overcome one or more disadvantages of current topical fungal treatments. In one aspect, the topical composition may include a redox gas solution.

In another aspect, a further exemplary embodiment provides a method of treating fungal infections like onychomycosis comprising contacting a skin or nail surface with a perfluorocarbon liquid and converting at least a portion of the perfluorocarbon liquid into a redox gas solution. In another aspect, such method includes the step of dissolving a redox gas in a perfluorocarbon liquid proximate the site of a fungal infection like onychomycosis to be treated. In another exemplary embodiment, the redox gas is formed during a non-thermal plasma treatment step.

In accordance with another exemplary embodiment, first and second treatment vectors for a fungal infection like onychomycosis are provided, wherein the first vector includes a redox gas formed as a result of a non-thermal plasma treatment step, and the second vector includes a redox gas solution.

Plasma-Generating Devices

In accordance with one exemplary embodiment, a plasma-generating device may create antimicrobial plasma species proximate a nail or skin area to be treated.

As used herein, the term “antimicrobial” means tending to destroy microbes, prevent their development, or inhibit their pathogenic action, and includes reference to, without limitation, antibacterial and antifungal properties.

Plasma is a gas-like phase of matter that typically contains many more reactive chemistry species than gas. A plasma-generating device turns electrical energy and a preselected gas (typically air, argon or helium) into electric fields, energetic electrons, and favorable chemistry for antimicrobial therapy.

There are multiple technologies that have been used for plasma generating devices at atmospheric pressure and temperatures. Non-thermal plasma gas at atmospheric pressure have been generated by microwave-induced plasma systems, dielectric barrier discharge (DBD), corona discharge, gliding arc discharge, and atmospheric pressure plasma jet. U.S. Pat. No. 7,572,998 is hereby expressly incorporated by reference herein in its entirety for all purposes. The '998 patent describes some representative, but not exclusive, plasma generators that may be useful.

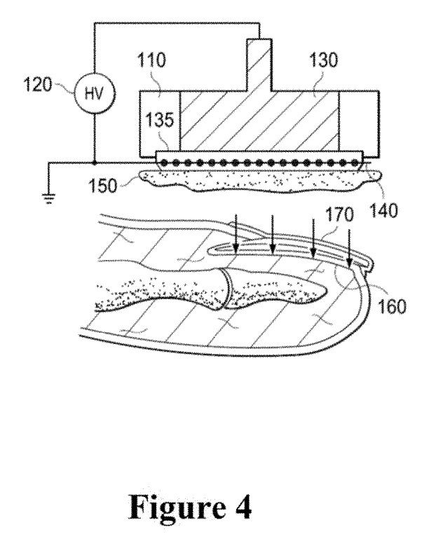

As shown in FIG. 4, the plasma-generating device 110 is electrically connected to both a power supply 120 and electrical control circuit to control both the duration and intensity of the plasma gas effluent 150. Generally, the nonthermal plasma subunit assembly comprises an electrical control circuit, a high voltage electrode, and a ground electrode. The electrical system may generate a high-voltage alternating current, typically between 2 to 20 kV at a frequency between 1 to 60 kHz. The power consumption may range from 0.05 W to 10.0 W. The high voltage electrode 130 may include a copper block covered by a thin quartz plate 135 (e.g., approximately 1 mm in thickness). A stainless-steel woven wire mesh may be positioned next to the quartz plate 135 and used as the ground electrode 140, A wire diameter of 0.5 mm and mesh density of 8×8 meshes per cm², for example, may be used.

A variation of the dielectric barrier discharge (DBD), the surface micro-discharge (SMD), may prove advantageous as the plasma-generating device in applications for igniting a stable plasma at ambient conditions. An SMD is a configuration of a DBD where the high voltage electrode is separated from a grounded electrode by a dielectric layer. The term “dielectric barrier discharge” may be used because a high electric field is generated through an electrical insulator (e.g., glass) to create a plasma. In an SMD, the charged particles may be confined to a plasma generating region around the grounded metal electrode. A SMD plasma device may be a desirable source of reactive chemical species for several reasons: the treated body part is electrically isolated from high-voltage electrode due to the ground electrode; the discharges are non-thermal, increasing adjacent gas temperature slightly, e.g., by only a few degrees; devices can be scaled simply, e.g., by changing electrode size and input power; and the discharges may operate in ambient air, e.g., without requiring a noble gas mixture. SMD devices have been further described in a series of articles by Graves et al. and Morfill et al.

Multiple reactive oxygen species (ROS) and reactive nitrogen species (RNS) may be generated in a non-thermal plasma. The active content of the plasma effluent at the treatment surface may include, for example, singlet oxygen (1O₂), hydroxide (OH), hydrogen peroxide (H₂O₂), ozone (O₃), nitric oxide (NO), nitrous oxide (N₂O), nitrogen dioxide (NO₂) and other excited molecules of air constituents that includes other reactive nitrogen and reactive oxygen species such as HNO₂, NO₃, HNO₃ and N₂O₅. Side reactions with these chemistries also may generate beneficial liquids and solids as the nail keratin and water in the nail can maintain antifungal properties long after the treatment is complete. Charged particles, electric fields and UV light (UV radiation) also may be generated by plasma. The ionized gases generally last for very short periods of time (e.g., less than a second), but free radicals and reactive oxygen and nitrogen species that are electrically neutral may last long enough to be effective—perhaps up to meters away from the source—in destroying fungus, bacteria and their spores. These free radicals denature critical lipid, protein and nucleic acid contents of the microbes, ultimately causing cell death. Research has demonstrated the effectiveness of plasma gas as well as nitrogen and oxygen free radicals such as ozone or hydrogen peroxide vapor or a combination of these in causing retarded fungal growth and fungal death. The byproduct of the plasma process is water (H₂O) and carbon dioxide (CO₂). Kogelschatz and co-workers have performed early studies on the discharge characteristics and chemistry of air DBDs. More recently Sakiyama et al., created a plasma chemistry model of a SMD device to determine the dynamics of the reactive neutral species it produces.

The non-thermal plasma gas effluent may be directed to a treatment site in two ways: direct mode or indirect mode. Direct mode (see FIG. 4) puts the treatment area within the visible plasma region discharge or plume, which may be between about 0.0 mm and about 5.0 mm for most devices. In direct mode, UV light, some charged particles and electric fields, in addition to reactive neutral species, may directly reach the treatment surface 160, to which a perfluorocarbon 170 may be applied. Indirect mode may have the treatment surface between about 5.0 mm and about 1.0 meter away. Thus, charged particles and electric field may have dissipated or decayed and may not reach the treatment surface. The indirect mode may benefit from a gas delivery system, wherein the plasma gas effluent may be directed to the treatment surface with directional venting, tubing, inline fans, connectors, ports, etc., for input, delivery and output of antimicrobial agent to the treatment surface. That is, simple diffusion or forced air flow may promote delivery of reactive species to the nail bed where the non-thermal plasma effluent or gas composition acts as an antimicrobial agent.

Treatment may occur for a predetermined or desired period of time. The antimicrobial agent may be applied for a sufficient time to achieve an effective killing of all or a portion of the fungus within the nail structure. For example, a sufficient time for application may be a time from about 10 seconds to about 4 hours. In one exemplary embodiment, antimicrobial agent may be applied for a time from about 1 minute to about 15 minutes. In yet another exemplary embodiment, antimicrobial agent may be applied for a time from about 5 minutes to about 20 minutes. In a further exemplary embodiment, antimicrobial agent may be applied for a time from about 30 minutes to about 1 hour. In other exemplary embodiments, the application of antimicrobial agent may be cyclical in nature, wherein an electrical control circuit cycles the device on and off, e.g., for a predetermined period of time (for example a 50% duty cycle (1 minute on /1 minute off) for a 30 minute to 1 hour treatment period).

### Gaseous Species

Other reactive gases not created by a non-thermal plasma generator may be useful in making redox gas solutions. Such other reactive gases may include any gas capable of directly causing or actively triggering a reaction that eradicates pathogens within or on the surface of a material (e.g., skin or nail) where the gas is also readily soluble in a perfluorocarbon fluid. Such gases include, for example, methanethiol, bromine, chlorine, nitric oxide, ozone, chlorine dioxide, and/or sulfur dioxide. Reactive oxygen and reactive nitrogen species play a central role in oxidation-reduction biochemistry (also called redox biology) and are active in the immune response of both animals and plants. The reactive oxygen/nitrogen/chlorine/bromine or sulfur species listed in Table 1 may be useful in making redox gas solutions.

TABLE 1
List of various reactive oxygen, nitrogen,
halogen and sulfur species [10, 22, 23].

| | |
|---|---|
| Radical | Non-radical |

Reactive oxygen species (ROS)

| | |
|---|---|
| Superoxide, O₂⁻ | H₂O₂ |
| Hydroxyl, OH | Ozone, O₃ |
| Hydroperoxyl, HO₂ | Singlet oxygen (O₂ 1 Dg) |
| Carbonate, CO₃⁻ | Hypobromous acid, HOBr |
| Peroxyl, RO₂ | Hypochlorous acid, HOCl |
| Alkoxyl, RO | |
| Carbon dioxide radical CO₂⁻ | Hypoiodous acid, HOI |
| Singlet (¹O₂) | Organic peroxides, ROOH |
| | Peroxynitrite, ONOO— |
| | Peroxynitrate, O₂NOO— |
| | Peroxynitrous acid, ONOOH |
| | Peroxomonocarbonate, HOOCO₂⁻ |
| | Carbon monoxide, CO |

Reactive chlorine/bromine species

| | |
|---|---|
| Atomic chlorine, Cl | Chloramines |
| Atomic Bromine, Br | Chlorine gas, Cl₂ |
| | Bromine gas, Br₂ |
| | Bromine chloride, BrCl |
| | Chlorine dioxide, ClO₂ |

Reactive nitrogen species (RNS)

| | |
|---|---|
| Nitric oxide, NO | Nitrous acid, HNO₂ |
| Nitrogen dioxide, NO₂ | Nitrosyl cation, NO⁺ |
| Nitrate radical, NO₃ | Nitroxyl anion, NO⁻ |
| | Dinitrogen trioxide, N₂O₃ |
| | Dinitrogen tetroxide, N₂O₄ |
| | Dinitrogen pentoxide, N₂O₅ |
| | Alkyl peroxynitrites, ROONO |
| | Alkyl peroxynitrates, RO₂ONO |
| | Nitryl chloride, NO₂Cl |
| | Peroxyacetyl nitrate, CH₃C(O)OONO₂ |

Reactive sulfur species

| | |
|---|---|
| Thiyl radical S. | Hydrogen sulfide, H₂S |
| | Disulfide, RSSR |
| | Disulfide-S-monoxide, RS(O)SR |
| | Disulfide-S-dixide, RS(O)2SR |
| | Sulfenic acid, RSOH |
| | Thiol/sulfide, RSR′ |

Redox gases can be purchased in their gaseous form, but due to their relatively high vapor pressure, require expensive sealed and pressurized tanks for storage. To provide handling convenience and cost-effectiveness, redox reagents are often created in an aqueous solution such as hypochlorous acid (generated from sodium hypochlorite), hydrogen peroxide, or nitric acid. These aqueous solutions are, however, less reactive than their gaseous counterparts, often requiring elevated temperatures and significant time to complete the redox reaction in situ.

A redox gas may be added to a perfluorocarbon liquid by any conventional technique (e.g., sparging or gas injection or simple diffusion) to create a redox gas solution. A typical sintered sparger can be purchased from Mott corporation (http://www.mottcorp.com). Such redox gas solutions provide a means for delivering a stable solution of an oxidation or reduction gas in its most active, non-hydrolyzed state that also allows handling convenience and cost-effectiveness.

Effective treatment of fungal nail infection may come from, combining a redox gas with a perfluorocarbon, then applying the redox gas solution topically, so that it penetrates the nail plate and inactivates pathogens residing in the nail bed. The non-thermal plasma generator to create reactive gases incorporates a permeable UV filter to mitigate damage to skin proximal to the infection from UV radiation (UVR). The permeable UV filter allows the device of the invention to be used to treat an infection for a longer duration of time, which increases the beneficial antimicrobial effects of the treatment due to a longer exposure time to the reactive species.

A method for treating nail fungus may comprise the steps of preparing a redox gas solution and applying the redox gas solution to the infected nails. An applicator may be used to coat the infected nail.

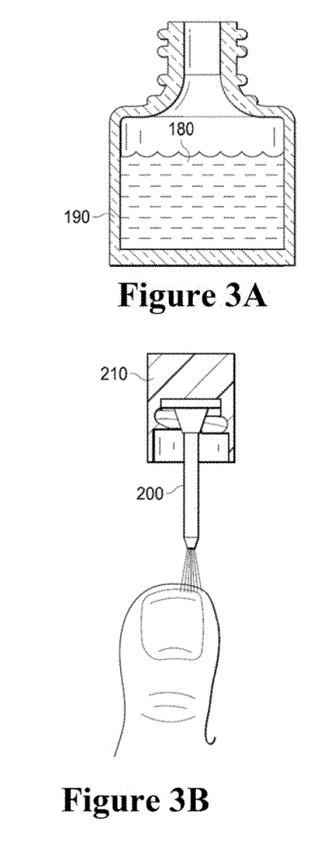

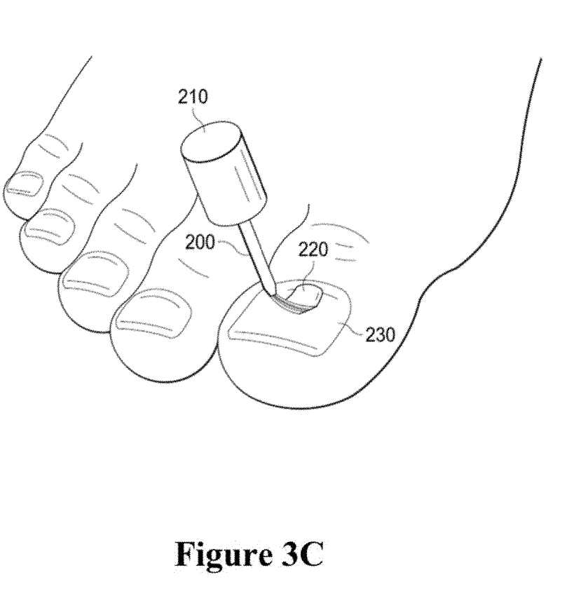

As shown in FIGS. 3A-3C, in one exemplary embodiment, a redox gas solution 180 may include chlorine dioxide gas dissolved in perfluorodecalin to saturation and may be provided in a container 190 as a topical treatment for onychomycosis. The solution may have a minimum of 80 ppm chlorine dioxide as the treating agent. Treating an infected nail with the solution comprises using an applicator 200 (for example coupled to the cap 210 of container 190) to spread a coating 220 on and around the nail 230. In one embodiment, such application includes a series of successive treatments to improve the aesthetic appearance of the nail, destroy the fungal infection and promote healthy nail growth. In another embodiment, the solution may be applied once a day for a minimum of sixty days to inactivate the fungus. In another embodiment, the solution may be applied once a week to prevent fungus or fungal spores from reinfecting the nail matrix.

A redox gas solution alternately may be generated at the skin or nail site by applying a perfluorocarbon liquid topically to the treatment area then treating the site with a redox gas, such as a gas created by a plasma-generating device. See FIGS. 2 and 4.

As an example, a perfluorocarbon liquid agent mixture may be administered immediately prior to treatment with a non-thermal plasma device in an amount sufficient to enhance the permeation of antifungal gas through and around the nail. The non-thermal plasma device allows the in-situ generation of gaseous reactive oxygen species and reactive nitrogen species that have antifungal properties.

The nonthermal plasma subunit 5000 shown in FIG. 15 is comprised of a High Voltage electrode 5100, Dielectric Barrier 5200, wire mesh ground electrode 5300 and permeable UV filter 5400. The permeable UV filter 5400 differs from the prior art because it is permeable to the plasma gas. The permeable UV filter 5400 is fabricated from Solar Screen fabric produced by Twitchell Corp. The permeable UV filter 5400 can be fabricated from either one or two or multiple layers of the Solar Screen fabric, Testing has shown that a single layer of the Solar Screen fabric blocks 75% of UVA and UVB light and a double layer of the Solar Screen fabric blocks 95%, and the Solar Screen fabric does not highly absorb the reactive plasma gasses generated by the nonthermal plasma subunit 5000 allowing the reactive gases to effectively treat the infection. The Solar Screen fabric used to form the UV filter 5400 can be enhanced by adding Titanium oxide to the UV filter 5400 and by doing so the UV blocking properties of the UV filter 5400 is improved. Additionally, the UV filter 5400 therapeutic properties can be enhanced by adding anti-fungal compounds such as Undecylenic acid, 5-Fluorocytosine nucleoside, Terbinafine hydrochloride, or Glycophosphate to the UV filter fabric as a protective barrier that protects the fabric from patient cross contamination and improves the fungal killing effectiveness of the instrument. The anti-fungal compounds can be coated onto the UV filter 5400 fabric by dip coating or spray coating.

The Titanium dioxide can be added to the UV filter 5400 by spray coating or dip coating the Solar Screen fabric and then drying it at low temperatures.

The anti-fungal compounds can be added to the UV filter 5400 fabric through a dip coating or spray coating. The preferred process is dip coating so that the anti-fungal compounds are attached to the surface of the UV filter 5400 fabric. Dip coating is a popular industrial coating process and is used to manufacture bulk products such as coated fabrics and specialized coatings in the biomedical field. Dip coating have been utilized in the biomedical field in the fabrication of bioceramic nanoparticles, biosensors, implants and hybrid coatings. For example, dip coating has been used to establish a simple yet fast nonthermal coating method to immobilize hydroxyapatite and TiO₂ nanoparticles on poly(methyl methacrylate) PMMA. A TiO₂ solution can be formulated by suspending the nanoparticles TiO₂ in an alcohol and glycol mixture or Titanium dioxide nanoparticles in monopropylene glycol or Titanium dioxide (TO₂-anatase) nanoparticles in neutral pH water. These formulations can also be purchased from Linari Nanotech (www.lanaribiomedical.com) product identifiers TiO₂-AG, TiO₂-MPG, TiO₂—WN.

Therefore, to mix the TiO₂nanoparticles into monopropylene glycol requires that significant energy be applied to the mixture similar to that found in U.S. Pat. No. 9,210,806, and is hereby incorporated by reference herein for all purposes. An example of a typical preparation of the mixture as shown in FIG. 13 is to take the 10 mg of TiO₂ nanoparticles powder 2000 from Milipore Sigma product number 637254 (http://www.sigmaaldirch.com) and placing it in a 300-ml suitable glass container such as a beaker 2010. Then add 200 ml of monopropylene glycol 2020 available from Vigo Ltd. Product number 94396 (http://www.vigoltd.com) into the beaker. Then place the sonicator tip of a Branson Sonifier 450 so that it extends to approximately 2 mm from the bottom of the beaker. Set the power supply for the controls accordingly to 30% Duty Cycle, Output control to 5 and timer to 9. Cover the beaker with a piece of plastic film such as Parafilm® M Sealing Film to prevent splatter or contamination and sonicate for 20 minutes. The sonification can also be completed in other devices such as placing the glass container in a Branson HT50 ultrasonic bath cleaner and sonicating the mixture until all the TiO₂ powder 2000 is suspended in the monopropylene glycol 2020 and is not in contact with the bottom of the glass container. Taking the mixture created and coating the permeable UV filter 5400.

In one embodiment, as shown in FIG. 16, a venting louver 6020 that enhances plasma treatment of nails is placed between the ground electrode of the plasma screen and the patient, thereby blocking some portion of the plasma screen but having holes 6025. The shape of the slot or holes of the venting louver can concentrate the plasma species over the nail or infected body part and improve fungal killing within the nail or infected body part. The louver divides the chamber into two parts, as shown in FIG. 16, which illustrates a 2-chamber system of the invention consisting of: The first sub chamber or reaction chamber 6000 that is the enclosed space underneath the SMD wire mesh ground electrode screen 6050 and the second sub chamber or treatment chamber 6100 which is configured to removably accept a patient object or body part to be antimicrobially treated. The two chambers (Reaction 6000 and Treatment 6100) are separated by the venting louver 6020 made of a plasma impermeable (and non-absorbable) barrier through which the generated long-lived plasma species 6300 can only enter the treatment chamber via open holes 6025. The holes can be covered with a permeable UV filter/blocking fabric 6030 to minimize the effect of UV exposure to the surrounding tissue near the infection. Therefore, UV radiation cannot enter the treatment chamber, but the long-lived plasma constituents i.e., reactive species 6300 can enter through holes 6025. The open holes 6025 can be variable in size or positioned in the venting louver 6020 as needed to best treat the object or body part. In the case of treating fungal nail, the holes in the barrier would be situated above the hallux (large) toenail region. By having a two-chamber system, the SMD plasma mesh surface area can be made as large as possible to maximize plasma output over time. However, because plasma reactive species 6300 takes longer to penetrate the nails than it does the skin, the barrier effectively concentrates the plasma as much as possible over the nails relative to the skin. This is achieved by applying the target area (nails) with perfluorodecalin liquid 6600 plasma absorbing material before treatment. However, the concentration of plasma over the hallux nail relative to the skin of the smaller toes and foot can also be done physically by limiting the generated plasma influx into the treatment chamber to the regions above the hallux nails. In other words, the holes 6025 in the barrier 6020 between the chambers would be made above the region in the treatment chamber 6100 where the hallux nail is expected to reside when the patient puts their foot into the treatment chamber 6100. Because the plasma species 6300 are passively diffusing and not under pressure, the plasma 6300 concentration in the treatment chamber will be highest in the vicinity of the barrier holes (i.e., above the hallux nail) and will be lower in regions farther away from the barrier holes (i.e., skin areas of the lesser toes and foot).

In one embodiment, the wire mesh ground electrode 6050 surface where the plasma is generated showed that the amount of plasma generated was increased by applying carbon nanotubes (CNTs) to the wire mesh ground electrode 6050. Experimentation showed that the plasma 6500 is strongest at edges such as in the corners of the wire mesh ground electrode 6050. Therefore, carbon nanotube deposition is a way to increase the number of corners on the wire mesh ground electrode and this would result in increasing the strength of the plasma 6500. Increased plasma strength further corrodes the wire mesh, which meant that there needed to be an improvement in corrosion resistance of wire mesh ground electrode 6050. One way to achieve corrosion resistance was theorized to be the addition of carbon nanotubes to the stainless steel wire mesh ground electrode 6050. To accomplish attaching CNTs to the stainless steel wire mesh ground electrode 6050, a means of applying the carbon nanotubes was needed. Researching methods to apply the CNTs to the stainless steel mesh of the ground electrode revealed that the CNT s could be grown directly on the surface of stainless steel alloys, because the native composition of stainless steel contains elements that seed CNT growth upon hydrocarbon exposure at elevated temperature. The method selected for growing carbon nanotubes onto stainless steel requires acid immersion or oxidation of the stainless steel in air to treat the surface prior to hydrocarbon exposure for CNT growth.

Direct Growth of Aligned Multiwalled Carbon Nanotubes on Treated Stainless Steel Substrates. Charan Masarapu and Bingqing Wei, Langmuir, 2007, 23 (17), pp 9046-9049, DOI: 10.1021/la7012232 is hereby expressly incorporated by reference herein in its entirety for all purposes. Additionally, Direct Synthesis of Carbon Nanotube Field Emitters on Metal Substrate for Open-Type X-ray Source in Medical Imaging, Amar Prasad Gupta, Sangjun Park, Seung Jun Yeo, Jaeik Jung. Chonggil Cho, Sang Hyun Paik, Hunkuk Park, Young Chul Cho, Seung Hoon Kim, Ji Hoon Shin, Jeung Sun Ahn, Jehwang Ryu, Materials 2017 10 (8), 878 is hereby expressly incorporated by reference herein in its entirety for all purposes.

Using the techniques of the Direct Growth of Aligned Multi-walled Carbon Nanotubes on Treated Stainless Steel Substrates, carbon nanotubes were grown onto the stainless steel mesh of the ground electrode. Subsequent testing of the stainless steel mesh of the ground electrode with the CNT's grown on the mesh resulted in improved corrosion resistance. The corrosion resistance was improved by a factor of two and unexpectedly the plasma production was increased by a factor of 3. The application of CNT to the stainless steel mesh ground electrode appears to increase the number of effective corners found on the stainless steel mesh. Doing this had the unexpected benefit of reducing corrosion of the mesh in addition to improving plasma creation, thus increasing concentration of antifungal gas species around the electrode.

In one embodiment, the addition of a chrome coating on the stainless steel mesh of the ground electrode showed increased performance in the effectiveness of the creation of the reactive species at the wire mesh ground electrode surface. A chrome coating on the stainless steel wire mesh produced two beneficial results; it reduced the corrosion of the mesh from reactive species degeneration at the ground electrode surface and it increases plasma creation. The chrome coating increased the concentration of gas in the treatment chamber due to the reduced reaction between the gas and mesh.

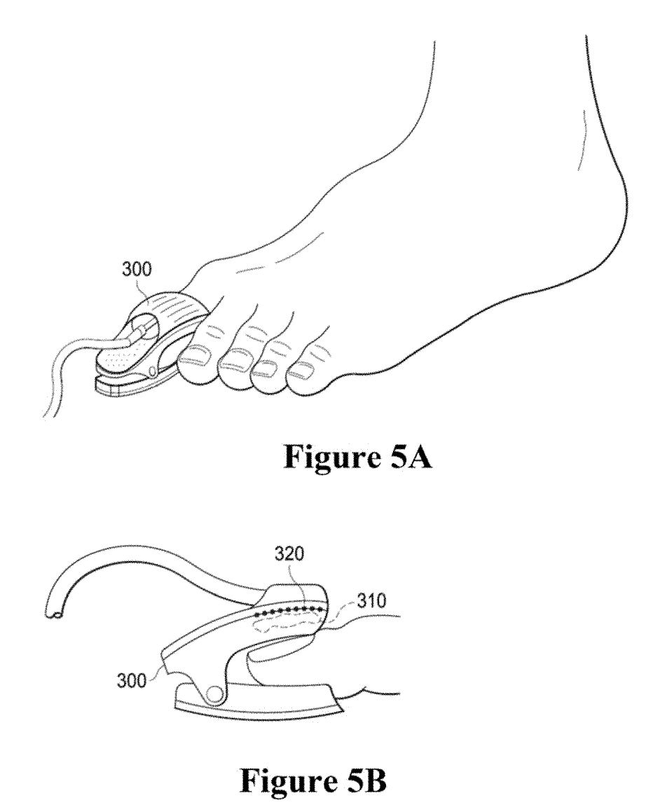

In one embodiment, perfluorodecalin liquid is pre-applied t the infected nail to act as a redox gas facilitator substance. As shown in FIGS. 5A and 5B, a toe-clip 300 incorporating a plasma-generating device 320 is attached to the infected toe and a 30-minute treatment protocol is initiated which generates antifungal gases 310 using electrical energy and air. This treatment may be performed as a series of successive treatments to improve the aesthetic appearance of the nail, destroy the fungal infection and promote healthy nail growth. The perfluorodecalin agent enhances the gas exchange between the nail bed and the plasma-generating device and allows the antifungal gas to more effectively penetrate the dense nail plate.

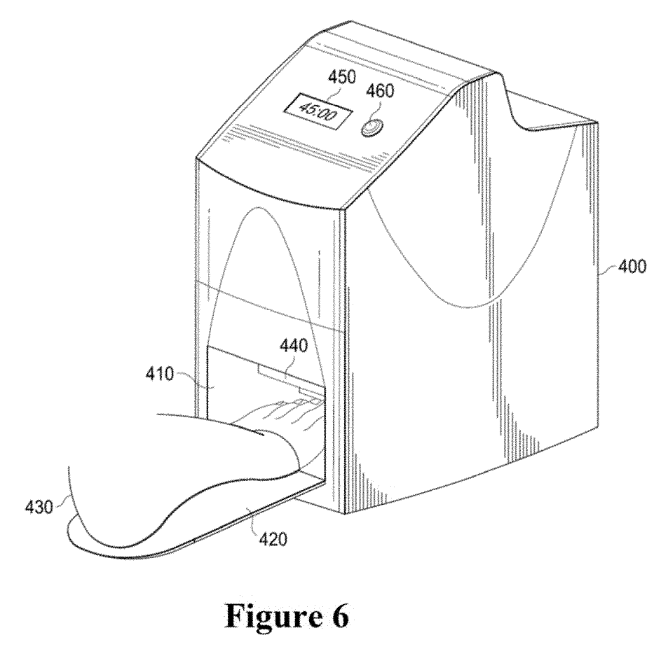

In another exemplary embodiment, a pre-made redox gas solution may be applied to a treatment area and additional treatment can be applied through the use of a non-thermal plasma treatment, thus replenishing or adding additional species of redox gas to the treatment area. As shown in FIG. 6, a plasma-generating device 400 may include a chamber 410 having a lid or door 420, within which chamber a foot 430 may be placed for treatment proximate a plasma source 440. The plasma may be provided for a predetermined amount of time using a control circuit including a timer 450 that is activated by pressing a start/stop button or switch 460. The duration time of the treatment is determined by setting a specific time for the amount of treatment on timer 450 and then removing said body part after said time has expired on timer 450.

In another exemplary embodiment, the method includes coating the affected nail( )along with the entire foot with the pre-made redox gas solution and subjecting the entire foot to curative gasses in order to destroy pathogens thereon and help prevent reinfection of the nail by pathogens residing elsewhere on the foot.

The redox gas solutions used during treatment may provide a means for delivering a stable solution of an oxidation or reduction gas in its most active, non-hydrolyzed state. In addition, the redox gas solutions may offer the advantage of providing a very low surface tension medium (generally on the order of approximately 15 dynes/cm), thereby enabling the oxidizing gas solution to efficiently contact and thoroughly penetrate a nail or skin infection, as well as reducing any associated inflammation.

Additionally, the PFC can act as a gas facilitator by providing an improvement in gas exchange between a non-thermal plasma device that creates in situ an antimicrobial gas and the nail barrier where the microbe resides. The PFC enhances the capacity of an active fungicidal gas to effectively penetrate the keratin matrix of fingernails and toenails such as to produce therapeutically relevant concentrations even in deeper regions of the matrix.

As shown in FIGS. 7-12, an exemplary embodiment of a system for treating onychomycosis is described. A main body housing 500 includes a chamber assembly 510, a power supply assembly 520, a shield assembly 530, an electronics panel assembly 540, and a rear cover assembly 550.

The main body housing 500 further may include an on/off button 560 for overall control of system operation. A display 570 disposed on the top of the main body housing 500 may provide desired information related to system operation. By way of example only, and without limitation, the display 570 may show a countdown timer reflecting the time remaining in a treatment session. Further, the main body housing 600 may include a handle 580 to help promote positioning of the system for a treatment session.

The chamber assembly 510 is shown in greater detail in FIG. 9. A chamber assembly housing 600 includes a chamber 610 disposed therein. A plasma head holder 620 disposed within the chamber generally positions a pair of plasma head assemblies 630 in the upper portion of the chamber 610. A plasma head assembly 630 may have a UV filter 640 disposed proximate the holder 620. The housing 600 includes an opening 650 at the lower front portion of the housing 600. A chamber hatch cover 660 may be attached to the housing 600 via a hinge above the opening 650, so that the cover 660 may be rotatable downwardly to a lower position covering at least a portion of the periphery of opening 650. A foot ramp 670 may be attached to the housing 600 via a hinge below the opening 660, so that during system non-use the ramp 670 may be rotatable upwardly to an upper position covering the cover 660 and opening 650.

In operation, the system includes use of a disposable liner 680. See FIGS. 9, 12A, and 12B. The liner 680 includes a tray portion 690 including a base 700, a back wall 710, and two side walls 720, 730. The liner 680 may be provided in a flat configuration (see FIG. 12A) and then be folded into a final configuration for use (see FIG. 128).

The base 700 proximately corresponds in size and shape to the size and shape of the bottom of the chamber 610. The front of tray portion 690 includes a perimeter support 740 and a film/covering 750. The support 740 and film/covering 760 are larger in peripheral size and shape as compared to the peripheral size and shape of the opening 650. In that way, the front of tray portion 690 may be held in place over the opening 650 and between the cover 660 and the front of housing 600 when the cover 660 is rotated into its lower position. In this position, the base 700 covers the bottom of chamber 610, and the back wall 710 and side walls 720, 730 cover at least part of the lower ends of the back and side walls of chamber 610; and a seal is provided to help prevent the escape of gases from the chamber 610 at the periphery of opening 650 during treatment.

The liner 680 may include a sheet 760 extending forward from the base of the front of tray portion 690. In one embodiment, the sheet 760 proximately corresponds in size and shape to the foot ramp 670. The sheet 760 may help to prevent contact between the treatment system and the foot 780 of a patient undergoing treatment. See FIG. 7.

As illustrated in FIGS. 7, 12A, and 12B, the film/covering 750 includes a port 800 therein. The port 800 may be sized and shaped so that a foot 780 may be inserted in part therethrough. In that way, the film/covering 750 acts as a seal about the foot 780 to help prevent the escape of gases from the chamber 610 during treatment.

To help position the foot within the chamber 610, a wedge 810 may be removably placed at the bottom of chamber 610 below the tray portion 690. For 24 mm high toes, a 4.5 degree wedge may be used. For 18 mm high toes, an 8.5 degree wedge may be used. In one embodiment, during treatment, the distance between the top of the toes and a plasma head assembly 630 may be approximately 15 mm. In one embodiment, the wedge 810 may be separate from the tray portion 690. In another embodiment, the wedge 810 may be formed with the tray portion 690, e.g., at the base 700.

In yet another embodiment, the wedge 810 may be formed by folding flat material to provide a wedge 810 of a desired size and shape to help position the foot within the chamber 610.

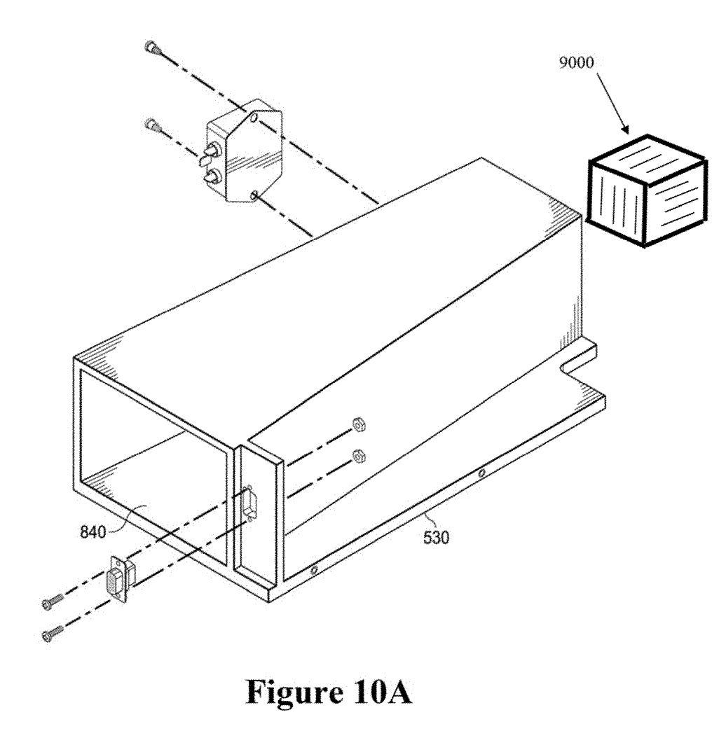

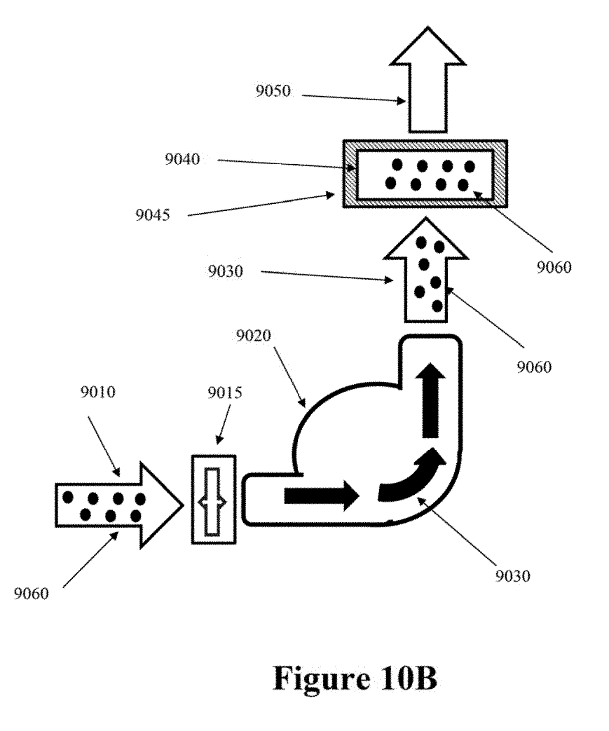

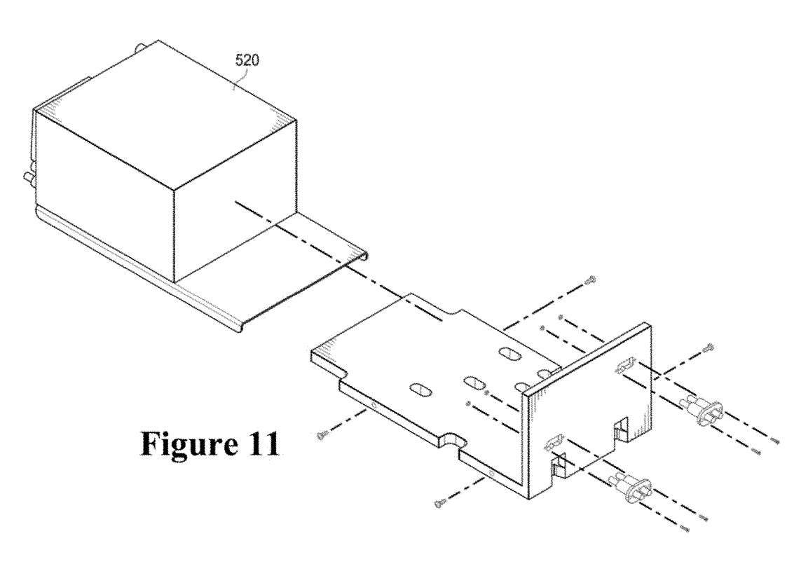

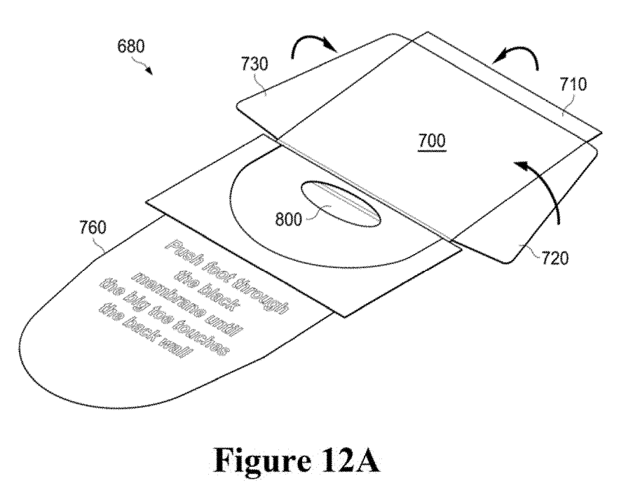

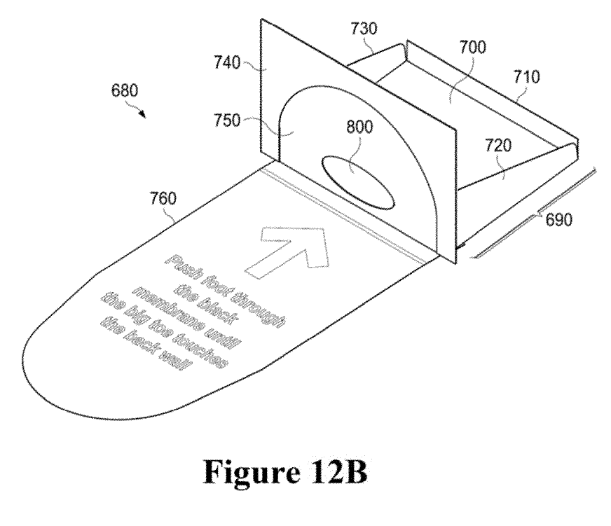

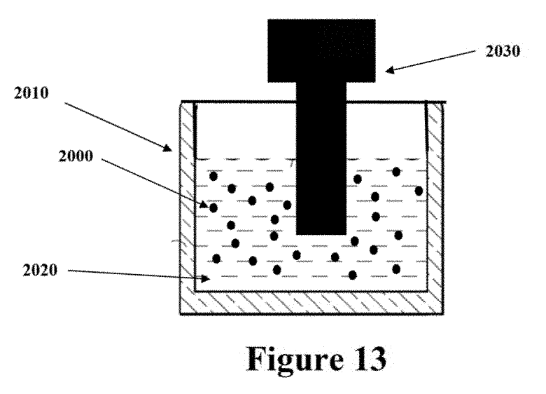

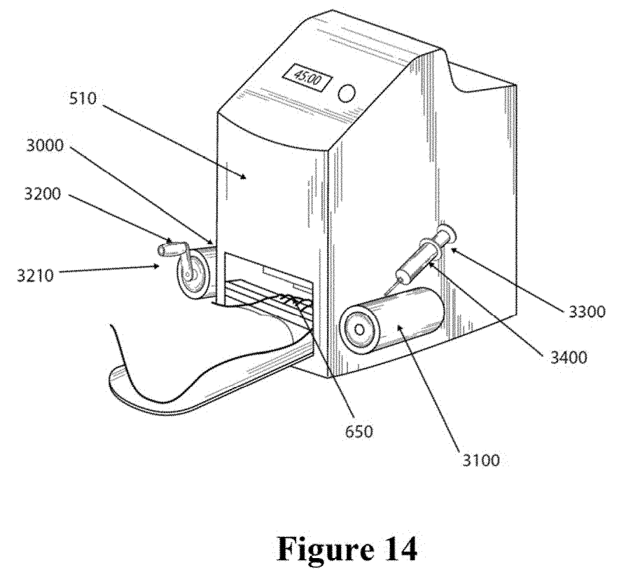

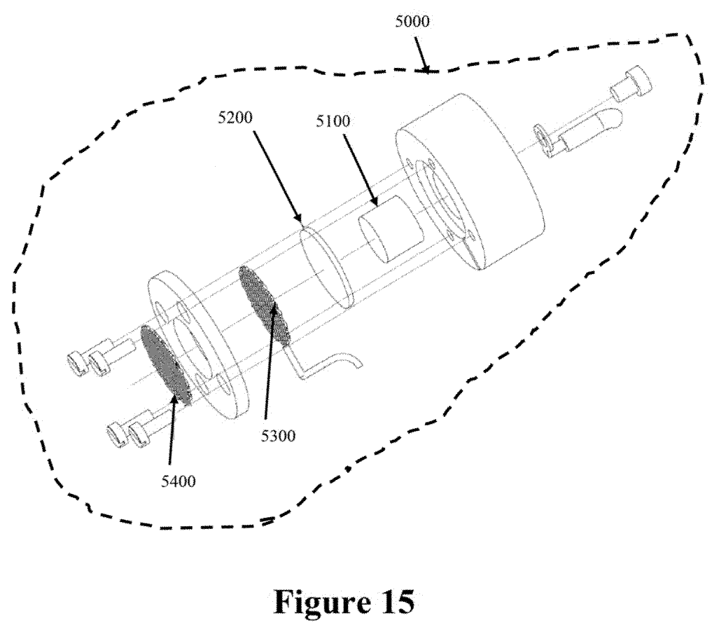

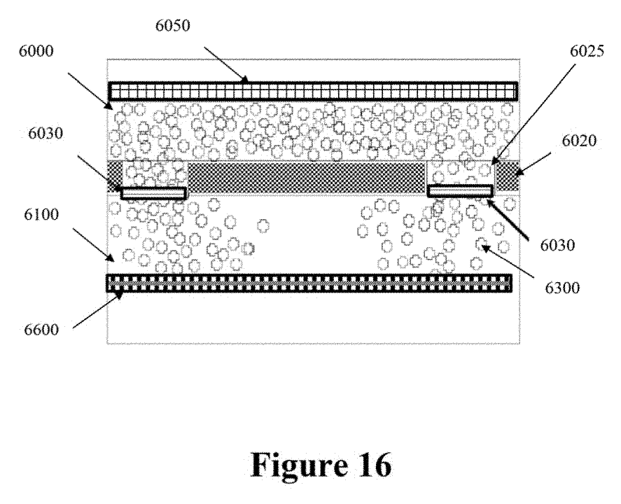

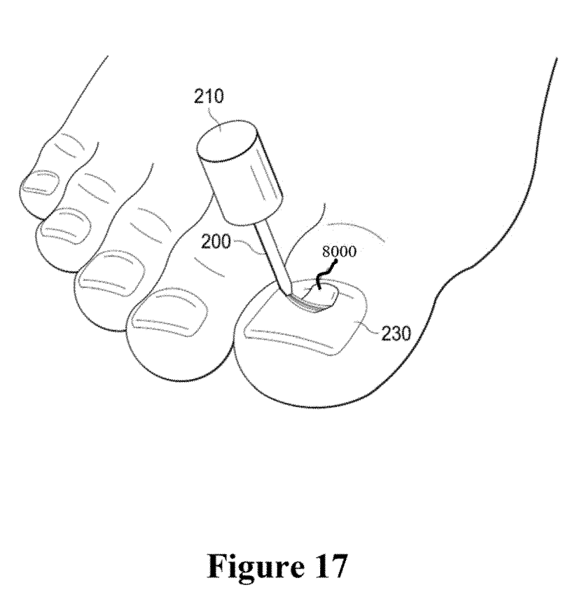

The rear wall of the, housing 600 may include an exhaust port 820. Mounted to the outside of housing 600 at exhaust port 820 may be an exhaust fan 830. In one embodiment, during the final 15 seconds of a treatment period the exhaust fan 830 turns on automatically to help empty the chamber 610 of any active gases. Activated gases may be directed by the fan 830 from chamber 610 through a carbon filter or a carboard filter or both disposed in exhaust duct 840 of shield assembly 530. See FIG. 10A. Deactivated gases then may exit the exhaust duct 840 at exit port 850 of rear cover assembly 550.

Using FIG. 10A and FIG. 10B, It is essential that plasma gasses from the treatment chamber 610 are captured during evacuation of the chamber 610 to eliminate toxic reactive species 6300 gasses which is shown as gas 9060 in FIG. 10A and 10B from escaping the device. The treatment chamber 610, when used to treat an infection, maintains high concentration of reactive species 6300 gas in the chamber during treatment. This high concentration of curative plasma gasses 6300 and beneficial anti-pathogenic substances generated that are needed to treat the infection must be captured at the end of the treatment to insure that they are not released into the atmosphere in the treatment facility/room. The preferred means of capturing the curative gasses and beneficial anti-pathogenic substances generated during treatment is to use a blower and filter cartridge 9000 which is shown in FIG. 10A. The filter assembly 9000 contains filter 9040 that can be formed from a pleated filter selected from appropriate materials such as a Pall membrane Breathing Circuit Filter BB50T made from Pleated hydrophobic membrane and having a large membrane surface area or an activated carbon filter or a combination of pleated and activated carbon filter and the filter is surrounded by cardboard support 9045. A circulating fan 9020 is currently used for drawing the curative plasma gases through the filter and clearing the reactive redox plasma gas concentrations from the chamber after treatment has ended. The curative gasses and beneficial anti-pathogenic substances are the gasses 9060 that need to be removed with the circulating fan air flow 9010. During the treatment procedure, the patient foot is inserted into the chamber 610 through a flexible gasket on the disposable, effectively preventing gasses from escaping and sealing the treatment chamber. Ventilation ports for removing the gas are sealed using at a minimum of one one-way silicon valve 9015 in the treatment chamber wall, which only allow gasses to leave when a pressure differential is applied by a fan 9020. Fan 9020 can also be a propeller style blade, squirrel cage blower, Axial-flow fans that have blades that force air to move parallel to the shaft about which the blades rotate, centrifugal fan similar to a squirrel cage blower, cross-flow fan, bellows, Coanda effect fan, or Electrostatic fluid accelerator. The evacuation fan 9020 comes on at the at least 15 seconds prior to the end point of the 45 minute treatment and stays on for an additional 1-2 minutes after the treatment is complete in order to ventilate the treatment chamber 610. The evacuation fan 9020 creates air flow 9030 within the chamber 610. The evacuation fan 9020 air flow 9010 opens the one-way silicon valves 9015 to create exhaust vents and that lowers the reactive species 9060 gas concentration shown by moving reactive redox plasma gas 9060 in the treatment chamber so that the patient can remove their foot without releasing reactive redox plasma gas 9060. The exhaust vent being capable of having at least one but not more than five exhaust valves 9015 to provide suitable exhaust vents. The silicon venting valves lead into an air duct 840 that has fan 9020 and three-stage activated carbon filter 9040 in a cardboard support 9045 all part of cartridge 9000 which limits the exposure of the device electronics to the reactive redox plasma gas by capturing the reactive redox plasma gas 9060 in the filter 9040. The reactive redox plasma gasses 6300 gasses which is shown as gas 9060 in FIG. 10A and 10B are exhausted through filter 9040 and captured in filter 9040 thus keeping the gas concentration levels below EPA environmental standards outside the instrument.

Testing of a matrix of materials showed that activated carbon filter 9040 and carboard support 9045 are both absorbers of reactive redox plasma gas produced by plasma. The carbon filter can have 1 to 5 stage carbon filter is made of a carbon filter at the exhaust fan. The preferred configuration is a 3-stage activated carbon filter such that a carbon filter sandwiched between two layers of perforated cardboard located in the air duct, and a carbon filter at the exhaust outlet of the cartridge. However, the activated carbon filter 9040 and cardboard support 9045 can be replaced by a pleated filter capable of capturing the gas 9060.

During operation, the plasma head assemblies 630 transfer their energy onto a patient's nail through diffusion of the plasma constituents. These constituents flow like a gas within the chamber 610 This action provides a controlled bathing of the nail in plasma energy at a low thermal profile. The typical temperature in the treatment chamber 610 may be 31-26° C. (89-79° F.) after a 45-minute treatment.

The disposable liner 680 may be single use only and help prevent cross contamination between patients by covering areas of the system that may come in contact with a patient's foot. The liner 680 includes a box-like tray portion 690 that may be made of medical grade APET plastic (polyester). The film/covering 750 and sheet 760 may be made of medical grade linear low density polyethylene, and may be latex free to avoid biocompatibility or allergy issues The disposable liner 680 may be locked in place in the system prior to insertion of a patient's foot through the opening or port 800 in film/covering 750.

Referring to FIGS. 2, 9, 14, the disposable transfer medium gas-permeable membrane 3000 is installed within the treatment chamber and on a dispensing reel 3100 and take-up reel 3200 and is exposed inside chamber 510. Dispensing reel 3100 includes a perfluorocarbon dispenser 3300 such as a syringe which dispenses perfluorocarbon liquid 3400 onto the membrane 3000. The membrane is positioned between one or more reactive species generators and the infected body part such as toes of the inserted foot or nails of the hand within chamber 510. With each insertion of infected body part the take-up reel 3200 is engaged by turning handle 3210 pulling membrane 3000 to position a clean section of membrane 3000 into chamber assembly 510. As the membrane 3000 advances past perfluorocarbon dispenser 3300 the operator dispenses perfluorocarbon liquid 3400 onto the membrane 3000 by depressing the plunger of the syringe of perfluorocarbon dispenser 3300 so that the perfluorocarbon liquid 3400 is imbibed or saturated into the membrane 3000. The membrane is then in position so that it contacts the infection on the patient's body part i.e., surface 90 of nail/tissue 100. During operation, the plasma head assemblies 630 transfer their energy onto a patient's infection through diffusion of the plasma via the perfluorocarbon liquid 3400 which has been applied to the membrane 3000 such that the perfluorocarbon liquid 3400 is imbibed or saturated into the membrane 3000 and is in communication with the infected body part. The plasma forms reactive species 60, 70. The reactive species 60, 70 dissolve in a perfluorocarbon liquid 80 applied to the membrane 3000 which is in communication with the surface 90 of nail/tissue 100. The redox gas solution including reactive species 60, 70 diffuses into the nail/tissue bed to eradicate fungus located therein. These reactive species 60, 70 flow like a gas within the chamber 610. This action provides a controlled bathing of the nail in plasma energy at a low thermal profile. The typical temperature in the treatment chamber 610 may be 31-26° C. (89-79° F.) after a 45-minute treatment.

Referring to FIG. 17, Treating an infected nail after treatment with reactive redox plasma gas 9060 by applying an acrylic or shellac coating 8000 using an applicator 200 (for example coupled to the cap 210 of container 190) to spread the acrylic or shellac coating 8000 on and around the nail 230. The reactive redox plasma gas 9060 treatment when an acrylic or shellac top coating 8000 is applied after the reactive redox plasma gas 9060 treatment to the nail increases the effectiveness of the treatment because nails retain antifungal properties from the reactive redox plasma gas 9060 treatment for 24-48 hours after a plasma treatment (de-gassing). A top coat of acrylic or shellac coating 8000 after the last treatment extends the de-gassing time by forcing the diffusion downward into the nail 230 structure and increases the effectiveness of the treatment.

For convenience only, and without limitation, reference is made herein to treatment of a foot. Of course, other areas may be treated, as well, e.g., the hand, an ear, a wound, etc. Accordingly, for some treatments, the perfluorocarbon coating will not be placed within a chamber, but will coat all or a portion of a chamber's walls.

For convenience only, and without limitation, reference is made herein to perfluorodecalin solution or perfluorocarbon solution. As defined in the instant invention, these solutions are selected from perfluorinated liquids including the following: perfluoropentane, perfluorohexane, perfluoroheptane, perfluorooctane, perfluorodecalin, perfluoromethylcyclohexane, perfluorotributyl amine, perfluorotriamyl amine, perfluoro-N-methylmoφholine, perfluoro-N-ethylmoφholine, perfluoroisopropyl moφholine, perfluoro-N-methyl pyrrolidine, perfluoro-1,2-bis(trifluoromethyl)hexafluorocyclobutane, perfluoro-2-butyltetrahydrofuran, perfluorotriethylamine, perfluorodibutyl ether, and mixtures of these and other perfluorinated liquids.

The invention can also be used to treat other parts of the body such as the ear to treat an ear infection. Here, the perfluorocarbon liquid provides the similar benefits as it does when treating a hand or foot infection by helping to reduce any inflammation to associated tissue. For example, when used to treat an inner ear infection, to help position plasma gas within the ear canal, an ear probe may be positioned over the plasma generator head. The ear probe may include a tapered, generally conical, concavely curved outer surface that may be formed of a soft thermal insulator such as foamed polyurethane. The ear probe may be adapted to engage the ear canal of a subject. Perfluorocarbon, e.g., perfluorodecalin solution, may be added to the ear chamber. A disposable protective cap may be placed over the ear probe that contains a UV filter screen for blocking UV light produced by the plasma device. The disposable protective cap both blocks UV light and helps to maintain hygiene. Upon activation, plasma gas comes out of the device, through the UV filter screen and fills up ear chamber for ear infection treatment. In one embodiment, during treatment, the distance between the ear's tympanic membrane and a plasma head assembly may be approximately 5 mm.

By way of further example, forming a wound chamber about a wound treatment site may enable the precise treatment of wounds with a plasma environment. A flexible, impermeable barrier may be secured to healthy skin about a wound to form a wound chamber. Upon enclosure of the wound within the chamber, a micro-environment may be created isolating the wound from the surrounding non-sterile environment. The wound chamber thus may serve as a precise plasma delivery platform, with perfluorocarbon liquid (e.g., perfluorodecalin) added to the wound.

### EXAMPLES

The following examples are offered to aid in a better understanding of the present invention. These examples are not to be construed as an exhaustive compilation of all embodiments of the present invention and are not to be construed as limiting the scope thereof.

Eradication of T. rubrum through a bovine hoof using Perfluorode alin (PFD) facilitator and an SMD plasma-generating device.

The in vitro test model uses a bovine, hoof disk, a surrogate nail model established in the literature. It is used along with a modified Franz-type diffusion cell to isolate the fungal contaminated side of the hoof in an enclosed chamber that ensures the treatment path is through the hoof barrier. An equal amount of T. rubrum is pipetted onto each hoof disc consisting of a 100 ul suspension. Each hoof disk is then placed fungus side down into the modified Franz cell and sealed with an O-ring. Each hoof is then plasma treated for 45 minutes where the average thickness of hoof disks is 0.35 mm. Eight hoof disks were treated as:
a. Control that is placed directly into the wash tube without any treatment
b. Two PFD-only hooves had 3 ul PFD pipetted directly onto the hoof opposite side from the fungus. This was allowed to sit for 45 minutes in the hood before placing in the wash tube.
c. Two Plasma treatment only hooves get just a 45 minute plasma is treatment (i.e., no PFD).
d. Three Plasma treatment with PFD hooves had 3 ul PFD pipetted directly onto the hoof opposite side from the fungus immediately before applying plasma treatment.

After treatment, each hoof went through a fungal collection protocol consisting of washing, dilution, plating and incubation. The results after seven days of incubation was a colony count of about 24,000,000 from the control hoof plate. The two hooves that were treated with PFD-only had colony reductions of 7% and 28% compared to control. The two hooves that were treated with 45 minutes of plasma only had colony reductions of 84% and 88% when compared to control. The three hooves that were treated with both PFD and 45 minutes of plasma treatment showed a colony reduction of 94% for one hoof and no colonies or 100% for the other two hoofs.

Application of Perfluorocarbon onto the Skin/Nail

The contacting of the infected area with the perfluorocarbon can occur by various means. The perfluorocarbon can be applied with a dropper, foam tip swab, a cotton tip swab, or a sponge applicator. It may be sprayed or squeezed on in a foam or gel formulation.

A further apparatus for contacting the, infected area with a perfluorocarbon consist of a gas-permeable membrane that includes perfluorocarbon liquid in its composition imbibed or saturated in the membrane and allows for the rapid, enhanced and uniform transfer of plasma reactive species between the plasma device and the infection. The contacting of the infection with the perfluorocarbon membrane (e.g., a dressing, bandage, patch, sleeve, toe cot, etc.) may define the treatment site. The gas permeable membrane composition comprises at least one material selected from ceramics, polymers, woven substrates, non-woven substrates, polyamide, polyester, polyurethane, fluorocarbon polymers, polyethylene, polypropylene, polyvinyl alcohol, polystyrene, vinyl, plastics, metals, alloys, minerals, non-metallic minerals, wood, fibers, cloth, glass, and hydrogels. In one embodiment, the membrane barrier is a silicone composition that contains an effective amount of perfluorocarbon and acts as a dressing. For nail treatment, the membrane may be manufactured in a toe or finger cot shape that may be slipped easily over the infected digit.

Loading Perfluorocarbon (PFC) with Antifungal Gas

A PFC may be loaded with antifungal gas in a manufacturing facility. Alternately, a home-use device may be provided so that a patient may “load” the PFC at their home, then apply it to their toenails The loading can be done with a plasma device and/or a pure gas canister (NO, ozone, H₂O₂, etc.) before treatment application by the patient. As described, there are other ways of loading the PFC with antifungal gas after it is applied to the infection site (e.g., besides a plasma device). In one embodiment, a pure gas canister (NO, ozone, H₂O₂, etc.) may be used. In another embodiment, a secondary chemical reaction may create the gas. Redox gases can be purchased in their gaseous form, but have relatively high vapor pressure, require expensive sealed and pressurized tanks for storage. To provide handling convenience and cost-effectiveness, redox reagents are often created in an aqueous solution such as hypochlorous acid (generated from sodium hypochlorite), hydrogen peroxide, or nitric acid. These aqueous solutions are, however, less reactive than their gaseous counterparts, often requiring elevated temperatures and significant time to complete the redox reaction in situ.

Again, a redox gas may be added to a perfluorocarbon liquid by any conventional technique (e.g., sparging or gas injection or simple diffusion) to create a redox gas solution. A typical sintered sparger can be purchased from Mott corporation (http://www.mottcorp.com). Such redox gas solutions provide a means for delivering a stable solution of an oxidation or reduction gas in its most active, non-hydrolyzed state that also allows handling convenience and cost-effectiveness.

### Sterilization and Other Therapies

Use of the described system and method also may promote wound sterilization and healing, may treat ear infections, may improve the sterilization of medical devices, may treat dental infections, may treat acne and various other dermatological infections, may promote bleeding cessation, may improve hand disinfection hygiene, or may treat skin, esophagus or colon cancer. It can also be used in veterinarian animal health for all such described applications.

In one embodiment, a method, of sterilizing or decontaminating an item comprises the steps of: (a) coating the item to be sterilized with a perfluorocarbon liquid layer to allow the perfluorocarbon liquid to come in close proximity with the item; (b) generating a gaseous plasma around the item, such that both the liquid perfluorocarbon and the exterior of item is exposed to reactive components of said plasma; and (c) maintaining the item in said plasma for a time period sufficient to allow the active sterilizing species generated from the plasma to effect sterilization and destroy any microorganisms present.

In another embodiment, decontamination of a liquid or gas may occur using a PFC including a reactive species. Examples include, without limitation, using the PFC/reactive species solution to eliminate microbes in blood with the aid of a dialysis machine, and to improve the sterilization of air using conventional methods.

It should be understood that the foregoing description is only illustrative of the invention. Various alternatives and modifications can be devised by those skilled in the art having the benefit of this disclosure, without departing from the invention. Accordingly, the invention is intended to embrace all such alternatives, modifications and variances.

Certain exemplary embodiments of the disclosure may be described. Of course, the embodiments may be modified in form and content, and are not exhaustive, i.e., additional aspects of the disclosure, as well as additional embodiments, will be understood and may be set forth in view of the description herein. Further, while the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention.

To the extent that the term “includes” or “including” is used in the specification or the claims, it is intended to be inclusive in a manner similar to the term “comprising” as that term is interpreted when employed as a transitional word, in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

We claim:

1. A method of treating an infection on a body part comprising:
a) applying a perfluorocarbon to a body part with an infection,
b) positioning said body part in a chamber,
c) Said chamber having venting louver,
d) Said venting louver having holes disposed in said venting louver passing through said venting louver,
e) Said chamber having a nonthermal plasma subunit,
f) Said nonthermal plasma subunit disposed within said chamber such that said body part is proximal to said venting louver and said venting louver is proximal to said nonthermal plasma subunit,
g) Said nonthermal plasma subunit assembly comprising an electrical control circuit, a high voltage electrode, a dielectric barrier, and a ground electrode,
h) Said nonthermal plasma subunit having a plasma gas permeable UV filter disposed proximate to said plasma head assembly.
i) Said gas permeable UV filter having TiO₂ compounds attached to the surface of said gas permeable UV filter,
j) Said gas permeable UV filter having anti-fungal compounds attached to the surface of said gas permeable UV filter,
k) Said plasma head assembly capable of forming a plasma reactive species proximate to said ground electrode,
l) Said plasma head producing a reactive species which is in communication with said perfluorocarbon by passing between said plasma head assembly through the said holes in said venting louver to the focus said reactive species on said infected body part,
m) Said chamber having at least one one-way valve,
n) Said chamber being in communication with an exhaust device which contains a filter and a fan,
o) Said fan in said exhaust device that is in communication with filter,
p) Said filter capable of capturing said plasma reactive species.

2. A body part of claim 1, where the body part is selected from the group consisting of hand, foot, toe and finger.

3. An anti-fungal compound or compounds attached to the surface of said gas permeable UV filter of claim 1, where the anti-fungal compounds is selected from the group consisting of Undecylenic acid, 5-Fluorocytosine nucleoside, Terbinafine hydrochloride or Glycophosphate.

4. A perfluorocarbon solution of claim 1, where the perfluorocarbon liquid is selected from the group consisting of fluoroheptanes, fluorocycloheptanes, fluoromethylcycloheptanes, fluorohexanes, fluorocyclohexanes, fluoropentanes, fluorocyclopentanes, fluoromethylcyclopentanes, fluorodimethylcyclopentanes, fluoromethylcyclobutanes, fluorodimethylcyclobutanes, fluorotrimethylcyclobutanes, fluorobutanes, fluorocyclobutanse, fluoropropanes, fluoroethers, fluoropolyethers, fluorotributylamines, fluorotriethylamines, perfluorohexanes, perfluoropentanes, perfluorobutanes, perfluoropropanes, sulfur hexafluoride, Methylperfluorobutylether (GransilSiW 7100) or Perfluoro(tert-butylcyclohexane).

5. The ground electrode of claim 1 formed from a wire mesh having the wire presenting a surface to said plasma reactive species and said wire mesh surface having carbon nanotubes on said surface of said wire mesh.

6. The venting louver of claim 1 where the hole are in the form of a slot.

7. The venting louver of claim 1 where the holes are round.

8. The chamber of claim 1 having between one to five one-way valves in communication with said exhaust device.

9. The fan of claim 1 is selected from the group consisting of a propeller style blade, squirrel cage blower, Axial-flow fan, centrifugal fan, cross-flow fan, bellows, Coanda effect fan, or Electrostatic fluid accelerator.

10. Said filter of claim 1 is selected from the group consisting of a pleated filter, activated carbon filter.

11. Said filter of claim 1 contain both of a pleated filter and an activated carbon filter.

12. Said activated carbon filter of claim one having one to five stages.

13. A device for treating an infection on a body part comprising:
a) A chamber,
b) Said chamber having a venting louver,
c) Said venting louver having holes disposed in said venting louver passing through said venting louver,
d) Said venting louver divides said chamber into two chambers a first sub chamber and a second sub chamber,
f) Said second sub chamber for positioning said body part with an infection in a chamber,
g) Applying a perfluorocarbon to a body part with an infection,
h) Said nonthermal plasma subunit assembly comprising an electrical control circuit, a high voltage electrode, a dielectric barrier and a ground electrode,
i) Said nonthermal plasma subunit having a plasma gas permeable UV filter disposed proximate to said holes in said venting louver,
j) Said plasma head assembly capable of forming a plasma reactive species proximate to said ground electrode,
k) Said plasma head producing a reactive species which is in communication with said perfluorocarbon by passing between said plasma head assembly through the said holes in said venting louver onto said infected body part,
l) Setting a timer for the treatment time,
m) Removing said body part after said time has expired,
n) Said chamber having at least one one-way valve,
o) Said chamber being in communication with an exhaust device which contains a filter and a fan,
p) Said fan in said exhaust device that is in communication with a filter,
q) Said filter capable of capturing said plasma reactive species.

14. A body part of claim 13, where the body part is selected from the group consisting of hand, foot, toe and finger.

15. A perfluorocarbon solution of claim 13, where the, perfluorocarbon liquid is selected from the group consisting of fluoroheptanes, fluorocycloheptanes, fluoromethylcycloheptanes, fluorohexanes, fluorocyclohexanes, fluoropentanes, fluorocyclopentanes, fluoromethylcyclopentanes, fluorodimethylcyclopentanes, fluoromethylcyclobutanes, fluorodimethylcyclobutanes, fluorotrimethylcyclobutanes, fluorobutanes, fluorocyclobutanse, fluoropropanes, fluoroethers, fluoropolyethers, fluorotributylamines, fluorotriethylamines, perfluorohexanes, perfluoropentanes, perfluorobutanes, perfluoropropanes, sulfur hexafluoride, Methylperfluorobutylether (GransilSiW 7100) or Perfluoro(tert-butylcyclohexane).

16. The ground electrode of claim 13 formed from a wire mesh having the wire presenting a surface to said plasma reactive species and said wire mesh having carbon nanotubes on said surface of said wire mesh.

17. The chamber of claim 13 having between one to five one-way valve on communication with said exhaust device,

18. The fan of claim 13 is selected from the group consisting of a propeller style blade, squirrel cage blower, Axial-flow fan, centrifugal fan, cross-flow fan, bellows, Coanda effect fan, Electrostatic fluid accelerator.

19. Said filter of claim 13 is selected from the group consisting of a pleated filter, activated carbon filter or both.

20. Said activated carbon filter of claim 19 having one to five stages
